(19) United States
(12) Patent Application Publication
(10) Pub. No.: US 2020/0061090 A1
(43) Pub. Date: Feb. 27, 2020

(54) AGENT FOR PREVENTING WORSENING OF INSULIN RESISTANCE OR AMELIORATING INSULIN RESISTANCE

(71) Applicant: B FOOD SCIENCE CO., LTD., Chita-shi, Aichi (JP)

(72) Inventors: Takumi TOCHIO, Chita-shi, Aichi (JP); Yoshihiro KADOTA, Chita-shi, Aichi (JP)

(21) Appl. No.: 16/498,563

(22) PCT Filed: Mar. 31, 2017
(86) PCT No.: PCT/JP2017/013849

§ 371 Date: Sep. 27, 2019

(51) Int. Cl. A61K 31/702; A61P 3/10

(52) U.S. Cl. CPC A61K 31/702; A61P 3/10

(57) ABSTRACT

[Problem]

To provide: a substance and a method both for effectively preventing the worsening of insulin resistance or for ameliorating insulin resistance; a method for treating diabetes, impaired glucose tolerance, obesity, hyperlipemia, arteriosclerosis, hypertension, a heart disease or metabolic syndrome; and a method for preventing these diseases.

[Solution]

An agent for preventing the worsening of insulin resistance or for ameliorating insulin resistance, which contains 1-kestose as an active ingredient. According to the present invention, it becomes possible to prevent the worsening of insulin resistance or ameliorate insulin resistance in a human or animal body conveniently and effectively without little concerning about adverse side effects or safety. According to the present invention, it also becomes possible to prevent or treat diabetes, impaired glucose tolerance, obesity, hyperlipemia, arteriosclerosis, hypertension, a heart disease or metabolic syndrome by preventing the worsening of insulin resistance or ameliorating insulin resistance in a human or animal body.

### TECHNICAL FIELD

The present invention relates to an agent for preventing worsening of insulin resistance or ameliorating insulin resistance, a food composition for preventing worsening of insulin resistance or ameliorating insulin resistance, a method for preventing worsening of insulin resistance or ameliorating insulin resistance, and a method for treating diabetes, impaired glucose tolerance, obesity, hyperlipemia, arteriosclerosis, hypertension, a heart disease, or metabolic syndrome and a method for preventing these diseases.

### BACKGROUND ART

Insulin is a peptide hormone secreted from β cells in islets of Langerhans of the pancreas. A main physiological action of insulin is to lower a blood glucose level. Glucose in the body is an important energy source but glucose at a high level reacts with proteins in vivo due to its high reactivity of the aldehyde group and causes glycation, thereby posing adverse effects (glucose toxicity) on the body, whereby a level thereof is maintained at a constant range by insulin.

Meanwhile, insulin resistance is a concept referring to a condition in which sensitivity to insulin is reduced and an insulin action corresponding to an insulin level is not obtained. When the insulin resistance advances, a blood glucose-lowering action diminishes for an amount of insulin in the body. A larger amount of insulin is required to maintain blood glucose within a normal range, and accordingly at an initial stage of insulin resistance, “hyperinsulinemia”, a high insulin level in blood, occurs.

Such an insulin resistance and hyperinsulinemia are reported to be causes of diseases such as diabetes, high blood pressure, and coronary artery diseases (Non Patent Literature 1). Under such circumstances, substances for ameliorating insulin resistance have been studied and developed, and for example Patent Literature 1 discloses an agent for ameliorating insulin resistance containing an active ingredient obtained by adsorbing a lemon fruit extract onto a styrene adsorbent resin and eluting the extract using an ethanol aqueous solution, and Patent Literature 2 discloses an agent for preventing and/or ameliorating insulin resistance containing α,α-trehalose as an active ingredient.

### CITATION LIST

### Patent Literature

### Patent Literature 1

Japanese Patent No. 5563181

### Patent Literature 2

Japanese Patent No. 5869882

### Non Patent Literature

### Non Patent Literature 1

GERALD M. REAVEN, DIABETES, VOL. 37, 1988, pp. 1595-1607

### SUMMARY OF INVENTION

### Technical Problem

However, even with the substances described in Patent Literatures 1 and 2, no substance or method for effectively preventing worsening of insulin resistance or ameliorating insulin resistance has been provided satisfactorily and accordingly the development of such a substance and method are in demand. The present invention is made in order to solve these problems and has an object to provide an agent for effectively preventing worsening of insulin resistance or ameliorating insulin resistance, a food composition for preventing worsening of insulin resistance or ameliorating insulin resistance, a method for preventing worsening of insulin resistance or ameliorating insulin resistance, and a method for treating diabetes, impaired glucose tolerance, obesity, hyperlipemia, arteriosclerosis, hypertension, a heart disease, or metabolic syndrome and a method for preventing these diseases.

### Solution to Problem

As a result of extensive studies, the present inventors found that 1-kestose enhances sensitivity to insulin and prevents the worsening of insulin resistance or ameliorates insulin resistance in all rats of healthy model, obesity model, and type 2 diabetes model. Then, each of the following inventions was accomplished based on these findings.

(1) An agent for preventing worsening of insulin resistance or ameliorating insulin resistance according to the present invention comprises 1-kestose as an active ingredient.

(2) An agent for preventing worsening of insulin resistance or ameliorating insulin resistance according to the present invention can be used for preventing or treating diabetes, impaired glucose tolerance, obesity, hyperlipemia, arteriosclerosis, hypertension, a heart disease, or metabolic syndrome.

(3) A food composition for preventing worsening of insulin resistance or ameliorating insulin resistance according to the present invention comprises 1-kestose as an active ingredient.

(4) A method for preventing worsening of insulin resistance or ameliorating insulin resistance according to the present invention comprises a step of letting human or animal take 1-kestose to thereby prevent worsening of insulin resistance or ameliorate insulin resistance in the human or animal.

(5) A method for treating diabetes, impaired glucose tolerance, obesity, hyperlipemia, arteriosclerosis, hypertension, a heart disease, or metabolic syndrome according to the present invention comprises a step of letting human or animal suffering from diabetes, impaired glucose tolerance, obesity, hyperlipemia, arteriosclerosis, hypertension, a heart disease, or metabolic syndrome take 1-kestose to thereby ameliorate insulin resistance in the human or animal.

(6) A method for preventing diabetes, impaired glucose tolerance, obesity, hyperlipemia, arteriosclerosis, hypertension, a heart disease, or metabolic syndrome according to the present invention comprises a step of letting human or animal having the potential to suffer from diabetes, impaired glucose tolerance, obesity, hyperlipemia, arteriosclerosis, hypertension, a heart disease, or metabolic syndrome take 1-kestose to thereby prevent worsening of insulin resistance in the human or animal.

### Advantageous Effects of Invention

1-Kestose, a type of oligosaccharide, contained in vegetables and grains such as onion, garlic, barley, and rye, is a substance with a long history of being eaten. Further, 1-kestose is not recognized to have toxicity in any of the mutagenicity test, acute toxicity test, subchronic toxicity test, and chronic toxicity test. 1-Kestose, according to these, can be considered extremely safe (Food processing and ingredients, Vol. 49, No. 12, p. 9, 2014). Furthermore, 1-kestose is highly water-soluble, has favorable sweetness similar to sugar, and, for this reason, can be taken conveniently everyday as it is or as a sweetener or easily added to various foods, pharmaceutical products and the like.

Thus, according to the present invention, the worsening of insulin resistance can be prevented or insulin resistance can be ameliorated in human and animals conveniently and effectively with little concern about side effects and safety. Further, according to the present invention, diabetes, impaired glucose tolerance, obesity, hyperlipemia, arteriosclerosis, hypertension, a heart disease, or metabolic syndrome can be prevented or treated by preventing worsening of insulin resistance or ameliorating insulin resistance in human and animals.

### BRIEF DESCRIPTION OF DRAWINGS

FIG. 1 is a bar graph showing serum insulin levels of rats taking 1-kestose in an intake amount of 0 to 2.71 g/kg of body weight (Groups A to E).

FIG. 2 is a bar graph showing blood glucose levels of rats taking 1-kestose in an intake amount of 0 to 2.71 g/kg of body weight (Groups A to E).

FIG. 3 is a line graph showing body weights during breeding period of rats taking a high-fat diet and rats not taking it, and rats taking 1-kestose and rats not taking it.

FIG. 4 is a bar graph showing plasma insulin levels of rats taking a high-fat diet and rats not taking it, and rats taking 1-kestose and rats not taking it.

FIG. 5 is a bar graph showing blood glucose levels of rats taking a high-fat diet and rats not taking it, and rats taking 1-kestose and rats not taking it.

FIG. 6 is a bar graph showing plasma insulin levels of diabetes model rats and its control rats, and rats taking 1-kestose and rats not taking it.

FIG. 7 is a bar graph showing blood glucose levels of diabetes model rats and its control rats, and rats taking 1-kestose and rats not taking it.

### DESCRIPTION OF EMBODIMENTS

Hereinafter, an agent for preventing worsening of insulin resistance or ameliorating insulin resistance, a food composition for preventing worsening of insulin resistance or ameliorating insulin resistance, a method for preventing worsening of insulin resistance or ameliorating insulin resistance, and a method for treating diabetes, impaired glucose tolerance, obesity, hyperlipemia, arteriosclerosis, hypertension, a heart disease, or metabolic syndrome and a method for preventing these diseases according to the present invention are described in detail.

“1-Kestose” is an oligosaccharide of trisaccharides consisting of 1 molecule of glucose and 2 molecules of fructose. 1-Kestose can be obtained by an enzyme reaction using an enzyme as disclosed in Japanese Patent Laid-Open No. 58-201980 with sucrose as a substrate. Specifically, first, β-fructofuranosidase is added to a sucrose solution and allowed to stand at 37° C. to 50° C. for about 20 hours to carry out an enzyme reaction, thereby obtaining a 1-kestose-containing reaction solution. The 1-kestose-containing reaction solution is subjected to a chromatographic separation method as described in Japanese Patent Laid-Open No. 2000-232878 to separate 1-kestose from other saccharides (glucose, fructose, sucrose, and oligosaccharides of 4 or more saccharides) and purify them to obtain a 1-kestose solution of high purity. Subsequently, the 1-kestose solution of high purity is, after concentrated, crystallized by a crystallization method as disclosed in Japanese Patent Publication No. 6-70075, thereby obtaining 1-kestose in the form of crystal.

Alternatively, as 1-kestose is contained in commercial fructo-oligosaccharide, such a product may be used as it is, or 1-kestose may be separated and purified from fructo-oligosaccharide by the method described above and then used. In other words, a 1-kestose-containing composition such as an oligosaccharide containing 1-kestose may be used as the 1-kestose of the present invention. When a 1-kestose-containing composition is used, a purity of 1-kestose is preferably 80 mass % or more, more preferably 85 mass % or more, and further preferably 90 mass % or more. Note that, in the present invention, the “purity” of 1-kestose refers to the mass % of 1-kestose when the total amount of saccharides is 100%.

1-Kestose is used by letting human and animals take. Examples of the intake method include a method of an oral intake of 1-kestose by human or animal as it is or in the form of a drink or food or a pharmaceutical product. Examples of the intake amount (dose) of 1-kestose include 0.04 g/kg or more of body weight daily. Such an intake amount may be taken not only once but also in several divided times per day. As demonstrated in examples to be described later, when 1-kestose of such an intake amount is taken by human or animal, sensitivity to insulin of the human or animal can be enhanced and accordingly the worsening of insulin resistance can be prevented or insulin resistance can be ameliorated.

In the present invention, “worsening of insulin resistance” refers to an increase in insulin resistance. Further, “preventing the worsening of insulin resistance” refers to suppression of an increase in insulin resistance. Furthermore, “ameliorating insulin resistance” refer to a decrease in insulin resistance.

Quantitative measurement of human insulin resistance can be carried out by a method known by a person skilled in the art as shown in the following (i) to (vii) as examples.

(i) glucose clamp technique (Defronzo R A et al., Am J Physiol, vol. 237, E214-E223, 1979),

(ii) steady state plasma glucose technique (Harano Y et al., J Clin Endoclinol Metab, vol. 45, pp. 1124-1127, 1977),

(iii) minimal model method (Bergman R N et al., Diabetes, vol. 38, pp. 1512-1527, 1989),

(iv) HOMA method (Matthews D R et al., Diabetologia, vol. 28, pp. 412-419, 1985),

(v) Fasting blood glucose level,

(vi) Fasting blood insulin level, and

(vii) Plasma insulin level at oral glucose tolerance test (Oimatsu Hiroshi et al., Journal of the Japan Diabetes Society, vol. 43, No. 3, pp. 205-212, 2000).

In the above measurement methods, when (iv) HOMA method is used, a blood insulin level and a blood glucose level at fasting of a subject are first measured and HOMA-IR is calculated by the following formula 1.

HOMA-IR=(fasting blood insulin level(μU/mL)×fasting blood glucose level(mg/dL))/405 Formula 1

An increase in HOMA-IR of the same subject means an increase in insulin resistance and a decrease in HOMA-IR means a decrease in insulin resistance. Accordingly, when an increase in HOMA-IR can be suppressed by the intake of 1-kestose, it can be determined that the worsening of insulin resistance has been prevented by the present invention. Further, when a level of HOMA-IR can be lowered by the intake of 1-kestose as compared with that before the intake thereof, it can be determined that insulin resistance has been ameliorated by the present invention.

Further, it can be generally determined that an HOMA-IR of 1.6 or less is “insulin resistance negative”, more than 1.6 and less than 2.5 is “insulin resistance positive suspected” and 2.5 or more is “insulin resistance positive” (SOIKEN Clinic Nishitani Masahito, SOIKEN Clinical Test project, Metabolism & Endocrinology Medicine, Glycometabolism & Insulin resistance, [online], [searched on Mar. 30, 2017], Internet <URL: http://www.soiken.info/clinical/area02/content01.html>). Thus, when a person having an HOMA-IR of 1.6 or less can maintain the HOMA-IR of 1.6 or less by the intake of 1-kestose, it can be determined that the worsening of insulin resistance has been prevented by the present invention. Further, when a person having an HOMA-IR of more than 1.6 can have a lower level of HOMA-IR by the intake of 1-kestose than that before the intake thereof, it can be determined that insulin resistance has been ameliorated by the present invention.

Additionally, in the above measurement methods, when (vi) Fasting blood insulin level is used, it can be understood that an increase in fasting blood insulin level of the same subject means an increase in insulin resistance, and a decrease in fasting blood insulin level means a decrease in insulin resistance. Accordingly, when an increase in fasting blood insulin level can be suppressed by the intake of 1-kestose, it can be determined that the worsening of insulin resistance has been prevented by the present invention. Further, when a fasting blood insulin level can be lowered by the intake of 1-kestose as compared with that before the intake thereof, it can be determined that insulin resistance has been ameliorated by the present invention.

Further, it can be generally determined that a fasting blood insulin level of 2 to 10 μU/mL is “insulin resistance negative”, more than 10 μU/mL and less than 15 μU/mL is “insulin resistance positive suspected”, and 15 μU/mL or more is “insulin resistance positive” (SOIKEN Clinic Nishitani Masahito, SOIKEN Clinical Test project, Metabolism & Endocrinology Medicine, Glycometabolism & Insulin resistance, [online], [searched on Mar. 30, 2017], Internet <URL: http://www.soiken.info/clinical/area02/content01.html>). Thus, when a person having a fasting blood insulin level of 2 to 10 μU/mL can maintain a fasting insulin level of 2 to 10 μU/mL by the intake of 1-kestose, it can be determined that the worsening of insulin resistance has been prevented by the present invention. Further, when a person having a fasting blood insulin level of more than 10 μU/mL can have a lower fasting blood insulin level by the intake of 1-kestose than that before the intake thereof, it can be determined that insulin resistance has been ameliorated by the present invention.

Note that a blood glucose level can be measured by a method known by a person skilled in the art such as glucose oxidase method. Conveniently, a glucose level can be measured using above commercial glucometers which utilize the measurement principle such as “Antsense III (Horiba, Ltd.)”, “Freestyle Light (Abbott Japan Co., Ltd.)”, and “CareFast (NIPRO CORPORATION)”.

An insulin level can also be measured by a method known by a person skilled in the art such as ELISA (enzyme-linked immunosorbent assay) method. Conveniently, the measurement can be carried out using commercial insulin measurement kits which utilize the measurement principle above such as “Mercodia Human Insulin ELISA Kit (Mercodia AB)”, “Levis Insulin-Rat (FUJIFILM Wako Shibayagi Corporation)”, and “YK060 Insulin ELISA (Yanaihara Institute Inc.)”.

Examples of the specific embodiments of an agent for preventing the worsening of or ameliorating insulin resistance of the present invention include a pharmaceutical product, a quasi-drug, a food additive, and health food products such as a supplement.

Dosage form of pharmaceutical products, quasi-drugs, and supplements containing 1-kestose is not particularly limited and a dosage form suitable for an administration method can be suitably selected. For example, in the case of oral administration, the dosage form can be a solid or liquid dosage form such as a powder, a tablet, a sugar-coated tablet, a capsule, a granule, a dry syrup, a liquid, a syrup, a drop, or a drink formula.

Pharmaceutical products, quasi-drugs, and supplements in each of the above dosage forms can be produced by a method known by a person skilled in the art. For example, in the case of a powder, 800 g of 1-kestose and 200 g of lactose are thoroughly mixed and subsequently 300 mL of 90% ethanol is added thereto and wetted. Subsequently, the wet powder is granulated and then air-dried at 60° C. for 16 hours, followed by regulating particle sizes to obtain 1000 g of a powder having a suitable size (a 1-kestose content of 800 mg/1 g). Alternatively, in the case of a tablet, 300 g of 1-kestose, 380 g of powdery reduced starch syrup, 180 g of rice starch, and 100 g of dextrin are thoroughly mixed and subsequently 300 mL of 90% ethanol is added thereto and wetted. Subsequently, the wet powder is extruded and granulated, followed by air-drying at 60° C. for 16 hours to obtain granules. Then, these granules are regulated to a particle size using a 850 μm-sieve, and subsequently 50 g of sucrose esters of fatty acids was added to and mixed with 470 g of the granules, followed by pressing tablets using a rotary tablet press (6B-2, manufactured by KIKUSUI SEISAKUSHO LTD.) to obtain 5000 tablets (a 1-kestose content of 60 mg/tablet) having a diameter of 8 mm and a weight of 200 mg per tablet.

Examples of the specific embodiments of a food composition for preventing or ameliorating insulin resistance of the present invention include processed food products such as a beverage, a dairy product, an edible granule, a paste, a seasoning, a sealed-pouch food, a baby formula, a fermented food, a preserved food, a processed fishery product, a processed meat product, and a processed grain product, a food additive, a health food product, and animal feed.

1-Kestose can be used to be added in the process of typical production of various drink and food products, food additives, and animal feed. 1-Kestose has a sweetness degree of 30 and its taste, property and processability are close to sucrose's ones. For this reason, various drink and food products, food additives, and animal feed can be produced by replacing a part or whole of sugar with 1-kestose in the production process of various drinks and foods to use it in the same manner as sugar.

It is reported that insulin resistance can be a factor of diseases such as diabetes, high blood pressure, and coronary artery diseases (Non Patent Literature 1). Additionally, insulin resistance is commonly seen among patients with impaired glucose tolerance, obesity (particularly visceral obesity), hyperlipemia, and low HDL cholesterolemia. Insulin resistance, based on this finding, is considered to have impacts not only on blood glucose level but also on blood pressure, cholesterol, and triglyceride metabolism. A condition, in which two or more of high blood glucose, high blood pressure, and dyslipidemia in addition to visceral fat obesity (visceral obesity and abdominal obesity) are observed at the same time, is called metabolic syndrome, and various abnormalities are considered occurring due to insulin resistance.

It is reported that pioglitazone, an insulin sensitizer, when administered to genetically obese and diabetic mice (yellow KK mice), notably ameliorates high blood glucose, lipid abnormality (hyperlipemia), hyperinsulinemia, and impaired glucose tolerance, and when administered to old obese beagle dogs with insulin resistance positive, decreases fasting blood glucose levels and fasting blood lipid levels and also suppresses an elevation in blood triglyceride level after meal (Ikeda H et al., Arzneimittelforschung., vol. 40, pp. 156-162, 1990).

Further, it is reported that when pioglitazone is administered for 4 weeks in a dose of 10 mg/kg of body weight/day to rats which are induced to have insulin resistance and high blood lipids, hyperinsulinemia, and high blood pressure by continuous drink of a 10% fructose solution, all symptoms are ameliorated (Masami S. et al., Jpn. J. Pharmacol., vol. 74, pp. 297-302, 1997). Furthermore, it is reported that when pioglitazone, an insulin sensitizer, or glimepiride, an insulin secretion promoter, is administered to patients with type 2 diabetes and a coronary artery disease to compare the effects on suppression of arteriosclerosis progress, such a progress suppression effect was confirmed in pioglitazone (Nissen S E et al., JAMA, vol. 299, pp. 1561-1573, 2008).

In other words, based on these reports on pioglitazone, it is considered that diabetes, impaired glucose tolerance, hyperlipemia, arteriosclerosis, hypertension, heart diseases, and metabolic syndrome can be prevented or treated by enhancing insulin sensitivity to prevent the worsening insulin resistance or ameliorate insulin resistance.

Based on the above findings, when human and animals suffering from diabetes, impaired glucose tolerance, obesity, hyperlipemia, arteriosclerosis, hypertension, a heart disease, or metabolic syndrome take 1-kestose to ameliorate insulin resistance, these diseases can be treated. Additionally, 1-kestose can be used for producing therapeutic pharmaceutical products for the above diseases.

Similarly, when human and animals having the potential to suffer from diabetes, impaired glucose tolerance, obesity, hyperlipemia, arteriosclerosis, hypertension, a heart disease, or metabolic syndrome take 1-kestose to prevent the worsening of insulin resistance, these diseases which the human and animals have the potential to suffer from can be prevented. Additionally, 1-kestose can be used for producing preventive pharmaceutical products for the above diseases.

Hereinafter, the present invention is described in reference to each example. Note that the technical scope of the present invention is not limited to the features demonstrated by these examples. Additionally, in the present examples, “1-kestose” used was a composition containing 1-kestose with a purity of 99 mass % (B Food Science Co., Ltd.).

### EXAMPLES

### <Example 1> Effect of 1-kestose on Rats Taking an Ordinary Diet (Healthy Model)

### (1) Breeding of Rats

Feed containing 1-kestose in such a way as to be 0, 0.5, 1, 2.5 and 5 mass % was prepared by CLEA Japan, Inc. on commission. The composition of feed is shown below.

<<Composition of Feed>> (Unit is Mass %)

Cornstarch: 39.7486, milk casein: 20, pregelatinized cornstarch: 13.2, granulated sugar or granulated sugar and 1-kestose: 10, purified soybean oil: 7, cellulose powder: 5, mineral mix: 3.5, vitamin mix: 1, L-cystine: 0.3, choline bitartrate: 0.25, tertiary butyl hydroquinone: 0.0014.

Forty SD rats (Japan SLC, Inc.) were divided into 5 groups of 8 rats each to be Groups A to E. Group A and Groups B to E had discretionary intake of feed without 1-kestose and 1-kestose-containing feed, respectively, while bred for 30 days. Breeding conditions included a temperature of 23±1° C., a light period of 12 hours (8:00 to 20:00) and a dark period of 12 hours (20:00 to 8:00).

Note that it is reported that a drug intake amount by rats can be converted into an intake amount by a human adult by the following formula 1 (paragraph [0065] in Japanese Patent Laid-Open No. 2014-526521, Shannon Reagan-Shaw et al., The FASEB Journal, Vol. 22, March 2007, pp. 659-661).

Daily 1-kestose intake amount by a human adult(g/kg of body weight)=Daily 1-kestose intake amount by a rat(g/kg of body weight)×6/37 Formula 1

Accordingly, using Formula 1, a daily 1-kestose intake amount by a rat was converted to that by a human adult. The results are shown in Table 1.

### (2) Measurement of Serum Insulin Level and Blood Glucose Level

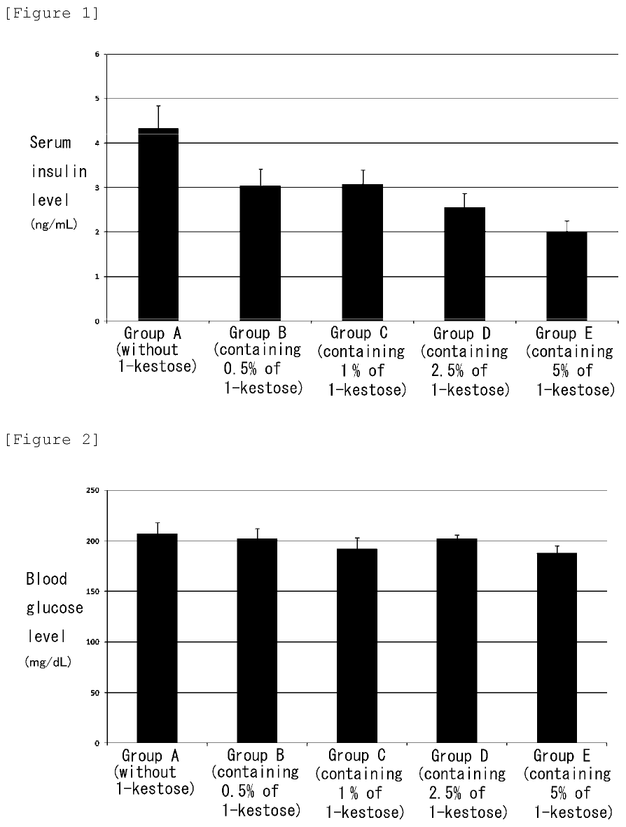

Blood was collected from the inferior vena cava of rats in each Group described in this Example 1 (1), centrifuged at 10000×g to obtain serum. The obtained serum was measured for an insulin level by ELISA technique and a glucose level by glucose oxidase method, to calculate an average value and a standard deviation by each Group. The results are shown in Table 1. Additionally, serum insulin levels and blood glucose levels are shown in FIG. 1 and FIG. 2 in bar graphs, respectively.

TABLE 1

| | | | | | Daily 1-kestose |
|---|---|---|---|---|---|
| | | | | | intake amount |
| | Serum | Blood | Daily feed | Daily 1-kestose | when rat is |
| | insulin | glucose | intake amount | intake amount | converted to |
| | level | level | (g/g of | (g/kg of | human (g/kg of |
| | (ng/mL) | (mg/dL) | body weight) | body weight) | body weight) |
| Group A | 4.34 ± 0.51 | 207 ± 11 | 0.05 | 0.00 | 0.00 |
| (without 1- | | | | | |
| kestose) | | | | | |
| Group B | 3.04 ± 0.38 | 202 ± 10 | 0.05 | 0.27 | 0.04 |
| (containing 0.5% | | | | | |
| of 1-kestose) | | | | | |
| Group C | 3.08 ± 0.31 | 192 ± 11 | 0 05 | 0.54 | 0.09 |
| (containing 1% | | | | | |
| of 1-kestose) | | | | | |
| Group D | 2.56 ± 0.31 | 202 ± 4 | 0.05 | 1.34 | 0.22 |
| (containing 2.5% | | | | | |
| of 1-kestose) | | | | | |
| Group E | 2.01 ± 0.24 | 188 ± 7 | 0.05 | 2.71 | 0.44 |
| (containing 5% | | | | | |
| of 1-kestose) | | | | | |

* p < 0.1 vs Group A,
** p < 0.05 vs Group A,
*** p < 0.01 vs Group A

As shown in Table 1 and FIG. 1, the serum insulin levels were significantly lower in Groups B, C, D, and E than Group A. Additionally, the serum insulin levels, when compared among each of Groups, were in the order of Group A>Group B≈Group C>Group D>Group E. In other words, the rats taking 1-kestose had lower serum insulin levels than the rats not taking 1-kestose. Further, the larger the 1-kestose intake amount was (contained amount of 1-kestose in the feed), the lower the serum insulin levels tended to be.

On the other hand, as shown in Table 1 and FIG. 2, blood glucose levels were equivalent among Groups A to E. In other words, the rats taking 1-kestose and the rats not taking 1-kestose had equivalent blood glucose levels. Additionally, the blood glucose levels were equivalent irrelevant to the intake amount of 1-kestose.

In other words, the rats taking 1-kestose and the rats not taking 1-kestose had equivalent blood glucose levels but the former had lower serum insulin levels. These findings revealed that the rats taking 1-kestose had enhanced insulin sensitivity. For this reason, these results revealed that the worsening of insulin resistance (an increase in insulin resistance) can be prevented by the intake of 1-kestose.

### <Example 2> Effects of 1-kestose on Rats Taking a High-Fat Diet (Obese Model)

### (1) Breeding of Rats

SD rats (Japan SLC, Inc.) were divided into 4 groups of 6 to 8 rats each to be Group A to Group D. During the breeding period, Group A and Group C had discretionary intake of tap water. On the other hand, Group B and Group D were allowed to take 1-kestose by providing discretionary intake of a solution in which 1-kestose was dissolved in purified water in such a way as to be 2 mass %. Additionally, Group A and Group B had discretionary intake of D12492J (Research Diets, Inc.) as an “ordinary diet”, and Group C and Group D had discretionary intake of D12492 (Research Diets, Inc.) as a “high-fat diet”. The rats were bred for 135 days with the same breeding conditions as Example 1 (1) except feed. The composition of feed is shown below.

<<Composition of Feed for Ordinary Diet (D12492J)>> (Unit is Mass %)

Casein: 200, L-cystine: 3, Cornstarch: 506.2, maltodextrin 10: 125, sucrose: 68.8, cellulose BW200: 50, purified soybean oil: 25, lard: 20, mineral mix S10026: 10, dicalcium phosphate: 13, calcium carbonate: 5.5, potassium citrate monohydrate: 16.5, vitamin mix V10001: 10, choline bitartrate: 2, yellow synthetic dye (FD&C Yellow Dye #1): 0.04, blue synthetic dye (FD&C Blue Dye #1): 0.01.

<<Composition of Feed for High-Fat Diet (D12492)>> (Unit is Mass %)

Casein: 200, L-cystine: 3, maltodextrin 10: 125, sucrose: 68.8, cellulose BW200: 50, purified soybean oil: 25, lard: 245, mineral mix S10026: 10, dicalcium phosphate: 13, calcium carbonate: 5.5, potassium citrate monohydrate: 16.5, vitamin mix V10001: 10, choline bitartrate: 2, blue synthetic dye (FD&C Blue Dye #1): 0.05.

As the average intake amount of water taken by a rat per day was about 22 mL/rat. From this result, it is considered that Group B and Group D took about 0.44 g/rat of 1-kestose daily.

### (2) Measurement of Body Weight, Plasma Insulin Level and Blood Glucose Level

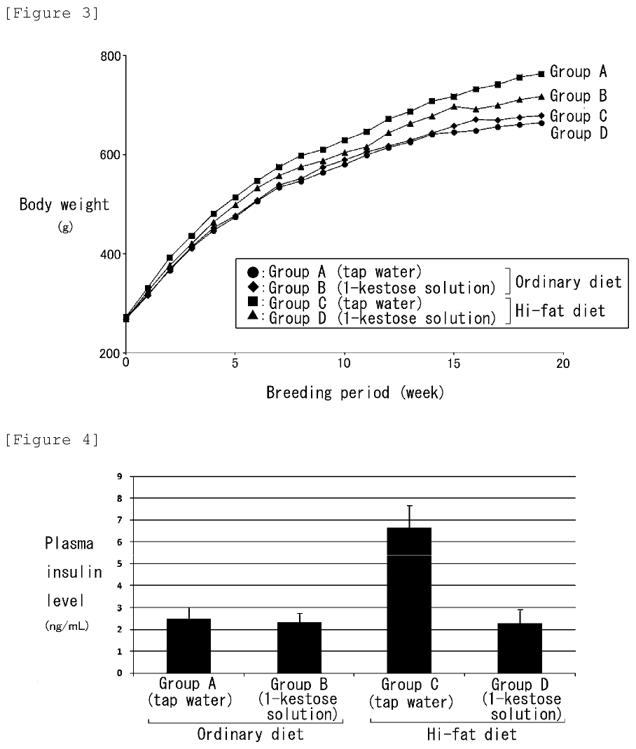

The rats of each Group in this Example 2 (1) were measured for body weight every week during the breeding period, and an average value by each Group was calculated and expressed in line graphs. The results are shown in FIG. 3. Note that the body weights (g) of each Group at the start of breeding period were 272±5 for Group A, 268±6 for Group B, 272±3 for Group C, and 269±3 for Group D.

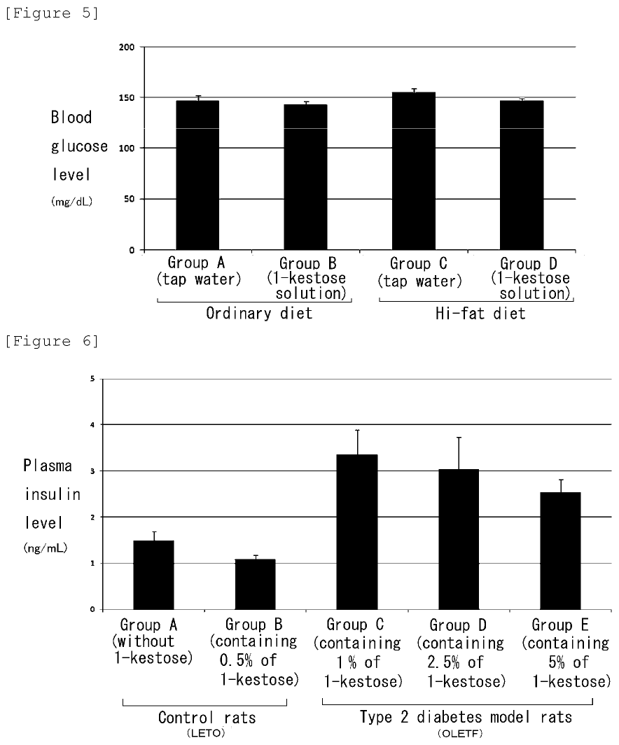

Further, plasma insulin levels and blood glucose levels were measured by the method described in Example 1 (2) to calculate an average value and a standard deviation by each Group. The results are shown in Table 2. Table 2 also shows average values and standard deviations of body weights at the end of breeding period (at the time when 19 weeks had passed). Additionally, insulin levels and glucose levels are shown in FIG. 4 and FIG. 5 in bar graphs, respectively.

TABLE 2

| | | | | | Daily 1- |
|---|---|---|---|---|---|
| | | Plasma | Blood | | kestose |
| | | insulin | glucose | Body | intake |
| | | level | level | weight | amount |
| | | (ng/mL) | (mg/dL) | (g) | (g/rat) |
| Ordinary | Group A | 2.48 ± 0.52 | 147 ± 5 | 665 ± 14 | 0.00 |
| diet | (tap water) | | | | |
| | Group B | 2.30 ± 0.42 | 143 ± 3 | 679 ± 22 | 0.44 |
| | (1-kestose | | | | |
| | solution) | | | | |
| High-fat | Group C | 6.65 ± 1.03 | 155 ± 4 | 764 ± 32 | 0.00 |
| diet | (tap water) | | | | |
| | Group D | 2.28 ± 0.63 | 147 ± 2 | 718 ± 21 | 0.44 |
| | (1-kestose | | | | |
| | solution) | | | | |

As shown in Table 2 and FIG. 3, the body weights at the end of breeding period were greater in Group C and Group D than Group A and Group B. In other words, when compared with the rats taking the ordinary diet, the rats taking the high-fat diet had greater body weights. From these results, it was considered obesity was caused by the intake of the high-fat diet.

On the other hand, as shown in Table 2 and FIG. 4, the plasma insulin levels were all equivalent in Group A, Group B, and Group D, and the plasma insulin level was notably higher in Group C than other Groups. In other words, the rats taking the high-fat diet and did not take 1-kestose had notably higher plasma insulin levels than the rats taking the ordinary diet. In contrast, the rats taking the high-fat diet and 1-kestose had equivalent plasma insulin levels to the rats taking the ordinary diet.

Further, as shown in Table 2 and FIG. 5, the blood glucose levels were equivalent in Groups A to D. In other words, the rats taking the ordinary diet and the rats taking the high-fat diet had equivalent blood glucose levels regardless of whether or not 1-kestose was taken.

In other words, the rats taking the high-fat diet and did not take 1-kestose had the obese condition with increased insulin resistance. In contrast, the rats taking the high-fat diet and 1-kestose did not have increased insulin resistance despite the occurrence of obese condition. Consequently, these results revealed that the worsening of insulin resistance (an increase in insulin resistance) observed in obese and diseases such as obesity can be prevented by the intake of 1-kestose.

### <Example 3> Effect of 1-kestose on Type 2 Diabetes Models

### (1) Breeding of Rats

OLETF (Otsuka Long-Evans Tokushima Fatty) rats (Japan SLC, Inc.), which are model animals with pathological conditions of type 2 diabetes, and LETO (Long-Evans Tokushima Otsuka) rats (Japan SLC, Inc.), which are the controls thereof, were prepared. The LETO rats were divided into 2 groups of 5 rats each to be Group A and Group B. Further, the OLETF rats were divided into 3 groups of 8 rats each to be Group C, Group D, and Group E. During the breeding period, Group A and Group C had discretionary intake of “feed without 1-kestose” described in Example 1 (1), Group B and Group E had discretionary intake of “feed containing 5 mass % of 1-kestose”, and Group D had discretionary intake of “feed containing 1 mass % of 1-kestose”. The rats were bred for 14 days with the same breeding conditions as Example 1 (1)

### (2) Measurement of Plasma Insulin Level and Blood Glucose Level

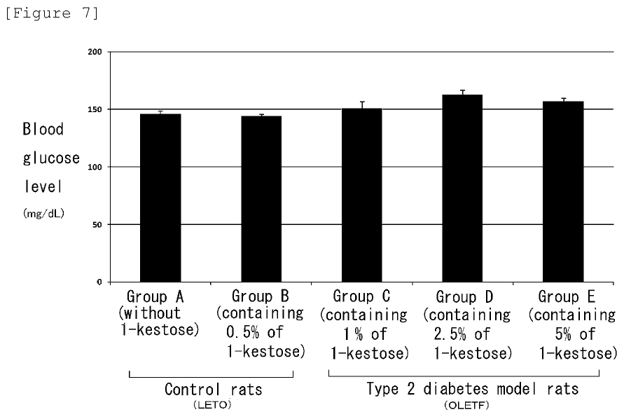

For the rats of each Group of this Example 3 (1), plasma insulin levels and blood glucose levels were measured by the method described in Example 1 (2) to calculate an average value and a standard deviation by each Group. The results are shown in Table 3. Additionally, plasma insulin levels and blood glucose levels are shown in FIG. 6 and FIG. 7 in bar graphs, respectively.

TABLE 3

| | | Plasma | Blood | Daily feed | Daily 1-kestose | Daily 1-kestose intake |
|---|---|---|---|---|---|---|
| | | insulin | glucose | intake amount | intake amount | amount when rat is |
| | | level | level | (g/g of | (g/kg of | converted to human |
| | | (ng/mL) | (mg/dL) | body weight) | body weight) | (g/kg of body weight) |
| LETO | Group A | 1.49 ± 0.19 | 146 ± 3 | 0.05 | 0.00 | 0.00 |
| | (without 1- | | | | | |
| | kestose) | | | | | |
| | Group B | 1.08 ± 0.09 | 144 ± 2 | 0.05 | 2.71 | 0.44 |
| | (containing 5% | | | | | |
| | of 1-kestose) | | | | | |
| OLETF | Group C | 3.36 ± 0.53 | 151 ± 6 | 0.05 | 0.54 | 0.09 |
| | (without 1- | | | | | |
| | kestose) | | | | | |
| | Group D | 3.04 ± 0.69 | 163 ± 4 | 0.05 | 1.34 | 0.22 |
| | (containing 1% | | | | | |
| | of 1-kestose) | | | | | |
| | Group E | 2.53 ± 0.29 | 157 ± 3 | 0.05 | 2.71 | 0.44 |
| | (containing 5% | | | | | |
| | of 1-kestose) | | | | | |

As shown in Table 3 and FIG. 6, the plasma insulin levels were higher in Group C, Group D, and Group E than Group A and Group B. In other words, the plasma insulin levels were higher in the OLETF rats than the LETO rats.

Further, in the comparison among the LETO rats, the plasma insulin levels were lower in Group B than Group A. In other words, the LETO rats taking 1-kestose had lower plasma insulin levels than the LETO rats not taking 1-kestose.

Furthermore, in the comparison among the OLETF rats, the plasma insulin levels were in the order of Group C>Group D>Group E. In other words, the OLETF rats taking 1-kestose had lower plasma insulin levels than the OLETF rats not taking 1-kestose, and the larger the intake amount of 1-kestose was, the lower the plasma insulin levels were.

On the other hand, as shown in Table 3 and FIG. 7, the blood glucose levels were equivalent in Groups A to E. In other words, the LETO rats and the OLETF rats had equivalent blood glucose levels regardless of whether or not 1-kestose was taken.

In other words, the OLETF rats had higher plasma insulin levels than the LETO rats while the blood glucose levels were equivalent. It follows from this that insulin resistance of the OLETF rats increased. Additionally, the OLETF rats taking 1-kestose and the OLETF rats not taking 1-kestose had equivalent blood glucose levels but the former had lower plasma insulin levels. This finding revealed that the OLETF rats taking 1-kestose had enhanced insulin sensitivity and thereby insulin resistance was ameliorated. For this reason, these results revealed that insulin resistance observed in diseases such as type 2 diabetes can be ameliorated by the intake of 1-kestose.

1-4. (canceled)

5. A method for treating diabetes, impaired glucose tolerance, obesity, hyperlipemia, arteriosclerosis, hypertension, a heart disease, or metabolic syndrome, comprising the following steps of (a) and (b);
(a) letting human or animal suffering from diabetes, impaired glucose tolerance, obesity, hyperlipemia, arteriosclerosis, hypertension, a heart disease, or metabolic syndrome take 1-kestose,
(b) ameliorating insulin resistance in the human or animal by the intake of 1-kestose of the step (a).

6. (canceled)

7. A method for producing an agent or a food composition comprising a step of producing an agent or a food composition that contains 1-kestose as an active ingredient and is used to prevent worsening of insulin resistance or ameliorates insulin resistance.

8. The method according to claim 7, wherein the agent or the food composition contains 1-kestose so as to be 0.04 g/kg body weight or more of daily intake.

9. The method according to claim 7, wherein the 1-kestose is an oligosaccharide containing 1-kestose and having a purity of 1-kestose of 80 mass % or more.

10. The method according to claim 7, wherein to prevent worsening of insulin resistance is to maintain a fasting blood insulin level within 2-10 μU/mL.

11. The method according to claim 7, wherein to ameliorate insulin resistance is to lower a fasting blood insulin level than that before the intake of 1-kestose.

12. A method for producing an agent or a food composition comprising a step of producing an agent or a food composition that contains 1-kestose as an active ingredient and is used to prevent or treat diabetes, impaired glucose tolerance, obesity, hyperlipemia, arteriosclerosis, hypertension, a heart disease, or metabolic syndrome.

13. The method according to claim 12, wherein the agent or the food composition contains 1-kestose so as to be 0.04 g/kg body weight or more of daily intake.

14. The method according to claim 12, wherein the 1-kestose is an oligosaccharide containing 1-kestose and having a purity of 1-kestose of 80 mass % or more.

15. The method according to claim 5, wherein 1-kestose is taken so as to be 0.04 g/kg body weight or more of daily intake by the human or animal in the step (a).

16. The method according to claim 5, wherein the 1-kestose of the step (a) is an oligosaccharide containing 1-kestose and having a purity of 1-kestose of 80 mass % or more.

17. The method according to claim 5, wherein ameliorating insulin resistance in the step (b) is lowering a fasting blood insulin level than that before the intake of 1-kestose.
